(19) United States
(12) Patent Application Publication
(10) Pub. No.: US 2005/0148005 A1
(43) Pub. Date: Jul. 7, 2005

(54) Dye solubilization binding assay

(76) Inventors: Pasha Emadi-Konjin, Richmond Hill (CA); Samad Talebpour, Richmond Hill (CA); Stephen W. Leonard, Unionville (CA); Tino Alavie, Thornhill (CA)

Correspondence Address: Ralph A. Dowell of DOWELL & DOWELL P.C., 2111 Eisenhower Ave., Suite 406, Alexandria, VA, 22314, US

(21) Appl. No.: 11/009,513

(22) Filed: Dec. 13, 2004

Related U.S. Application Data
(60) Provisional application No. 60/528,714, filed on Dec. 12, 2003.

(51) Int. Cl. C12Q 1/68; G01N 33/553

(52) U.S. Cl. 435/6; 436/526

(57) ABSTRACT

The present invention provides a method of conducting an assay for the detection of a target analyte with enhanced sensitivity, dynamic range, detection limit, selectivity and accuracy using a sandwich assay format. A liquid sample is first brought into contact with a solid phase, where the solid phase is coated with receptors that have a high affinity for an analyte that may be present in the sample. After an incubation period in which the analyte binds to the receptors, and is thereby immobilized onto the solid phase, a colloidal solution of dye particles is introduced. The dye particles are coated with a second type of receptor that also has a high affinity for the analyte, but a low affinity for the first receptor and also a low affinity for the solid phase. The dye particles therefore bind to the analyte and become immobilized onto the solid phase. The solid phase is then separated from the liquid phase, which in turn separates the bound dye particles from the unbound dye particles. A solubilization buffer, maintained at an appropriate pH, is then added to solubilize the bound dye particles, creating a dye solution. The fluorescence of the resulting dye solution is measured, wherein the solubilized dye molecules strongly absorb excitation light and emit light with high efficiency, and the concentration of the analyte is determined using a pre-determined standard curve.

### CROSS REFERENCE TO RELATED U.S. PATENT APPLICATIONS

This patent application relates to U.S. provisional patent application Ser. No. 60/528,714 filed on Dec. 12, 2003 entitled DYE SOLUBILIZAITON BINDING ASSAY, which is incorporated herein by reference in its entirety.

### FIELD OF THE INVENTION

The present invention relates to binding assays, in particular immunoassays, where a target analyte is bound to a capture agent via specific forces. The colourimetric and fluorometric assays disclosed herein employ the solubilization of bound dye particles for the amplification of the detected optical absorbance or fluorescence signal and the control of the dye particle radius for optimization of sensitivity and dynamic range.

### BACKGROUND OF THE INVENTION

Binding assays have found widespread use in the detection and quantification of analytes in a multitude of industries. The success of binding assays over alternative assay methodologies is owed to their ability to provide rapid, selective, sensitive and quantitative detection of a wide array of target species, ranging from small molecules to complex cellular antigens.

The most commonly used binding assay is the immunoassay, in which antibodies are employed to bind and immobilize a target analyte. Antibodies are protein molecules that are produced by an organism for the purpose of identifying and isolating an antigen that may pose a danger to the host organism. Antibodies have unique chemical and spatial structures designed to bind to target antigens with high affinity and specificity. This property of an antibody can be exploited to produce a highly specific and sensitive assay for a target analyte. For example, antibodies adsorbed onto a surface will capture and immobilize target analyte present in a sample. Although polyclonal and monoclonal antibodies provide an excellent means of capturing a target analyte, alternative binding assay receptors exist. These capture agents include oligonucleotides, phage display of antibody fragments, bacterial display of peptides and proteins, molecular imprinted polymers (MIP) and aptamers.

Regardless of the type of receptor used in a binding assay, a reporter or label agent is required to detect and measure the presence of the bound analyte. A wide variety of label technologies have been applied to immunoassays, enabling detection via optical, electronic, chemical or physical phenomena.

Although radioactive tracer labels were initially used for immunoassays, the hazardous nature of the radioactive isotopes hampered their adoption in widespread rapid testing. Enzymes have enjoyed remarkable success as immunoassay reporter molecules due to their ability to catalyze chemical reactions that produce large measurable signals. Since an enzyme is not consumed in a chemical reaction, it is capable of producing numerous reaction products from a single binding event. This multiplicative feature of enzyme-based immunoassays provides amplification and offers increased sensitivity. Following the catalysis of a chemical reaction in the presence of a bound enzyme, optical phenomena including chromogenesis (wavelength-dependent absorption), chemiluminescence, or fluorescence can occur. The most commonly used enzymes are horseradish peroxidase or alkaline phosphatase. Fluorescent molecules (fluorophores) are also often used as reporter molecules in immunoassays. Although fluorescence offers the ability to measure very low analyte concentrations with wide dynamic range, results are often compromised by a large background signal caused by autofluorescence (endogenous sample fluorescence), light-scattering effects, and non-specific binding.

The flexibility provided by the different labeling technologies has led to the development of many immunoassay formats for rapid screening and clinical testing. The most widely used immunoassay is the enzyme-linked immunosorbent assay (ELISA), which is typically a two-site binding or “sandwich” assay. Antibodies bound to a solid surface capture and immobilize antigens, which then capture a second antibody that is labeled with an enzyme. The enzyme catalyzes a reaction with a chromogenic substrate that leads to wavelength-dependent absorption, producing a colour change. Alternatively, the enzyme can catalyze a chemiluminescent reaction. ELISA offers inexpensive assays incorporating amplification, but with the disadvantages of requiring multiple wash steps, temperature dependence, poor repeatability owing to its multi-step nature, problems with reagent consistency, demanding storage requirements, and long incubation times.

Other assay formats involving fluorescence include phase-resolved fluorescence (PRF), time-resolved fluorescence (TRF), and fluorescence polarization (FP). The first two of these methods exploit fluorophores with very long spontaneous emission lifetimes. For example, in time-resolved fluorescence, the sample is subjected to a pulsed light source and the fluorescence signal is integrated only after waiting for the autofluorescence signal to decay. This provides a means of isolating the primary fluorescence (autofluorescence) from the desired secondary fluorescence signal, increasing the signal-to-noise ratio. Typical fluorophores used in such assays are lanthanide chelates such as europium, samarium, terbium and dysprosium. Although time- and phase-resolved fluorescence assays provide enhanced sensitivity and shorter acquisition time relative to ELISA, existing methods do not incorporate a means of amplification. Unlike temporally sensitive fluorescence assays, fluorescence polarization assays detect the polarization of light emitted by fluorophore labels. If a labeled molecule is bound through an antigen-antibody interaction, it is unlikely to undergo rotation upon the absorption of excitation light. This causes the emitted fluorescence to be polarized, allowing for the discrimination of bound and unbound fluorophores based on the degree of polarization of the collected fluorescence. Unfortunately, FP assays, which do not provide amplification, are only suitable for small analyte molecules since large molecules in solution are less likely to rotate and act as a source of polarized background fluorescence.

Each of the above immunoassay technologies possesses deficiencies related to one or more of the following effects: long and laborious process steps, inconsistency in producing reagents, poor signal amplification, large background, difficulty in storage, complexity in instrumentation, poor accuracy and long incubation or acquisition times. What is required is an assay format that provides an amplified detection scheme without the drawbacks listed above. A step towards this goal was recently achieved by Trau and coworkers, who described a novel immunoassay format employing nanoencapsulated microcrystalline particles for large amplification in a fluorescence assay (D. Trau et al., DE10042023 (2003), D. Trau et al., “Nanoencapsulated Microcrystalline Particles for Superamplified Biochemical Assays”, Anal. Chem. 74, 5480 (2002)). Their method involves a sandwich assay using antibody-coated nanoencapsulated crystalline fluorogenic precursor particles as labels. Such fluorogenic precursor label particles are capable of producing a very large number of dye particles upon solubilization, dramatically amplifying the measured fluorescence signal relative to that of an assay with a directly labeled fluorophore. The method offers an improvement over prior attempts at fluorophore amplification that were plagued by experimental difficulties and high cost, such as liposome encapsulation (H. A. Rongen et al., “Liposomes and Immunoassays”, J. Immunol. Methods 204, 105 (1997)) and perylene microparticles prepared via precipitation in an antibody-rich solution (A. Kamyshny and S. Magdassi, “Chemiluminescence Immunoassay in Microemulsions”, Colloids Surf. B 11, 249 (1998)). Unfortunately, the method requires that the microcrystalline particles are coated using a complex and labor-intensive “layer-by-layer” procedure for sufficient encapsulation and colloidal stabilization. Furthermore, the microparticles exist in a wide distribution of sizes, ranging from˜100 nm to 1.5 μm, producing non-optimal binding, washing and amplification. Finally, the solubilization of the dye is done in dimethyl sulfoxide, a hazardous solvent that may limit the usefulness of the approach.

A simpler and more effective approach to amplification through solubilization involves the use of colloidal dyes. Such dyes, also known as textile dyes, are non-toxic, inexpensive and widely available. Their detailed chemistry is well known and may be tailored for the attachment of a wide variety of antigens, antibodies and aptamers. Furthermore, they have excellent optical properties including strong visible absorption and efficient fluorescence (upon solubilization). Many different dyes exist with a broad range of colours for assay multiplexing. The use of colloidal dyes in immunoassays was pioneered by Gribnau and coworkers (T. C. J. Gribnau et al., U.S. Pat. No. 4,373,932 (1983), T. Gribnau et al., “The Application of Colloidal Dye Particles as Labels in Immunoassays: Disperse(d) Dye Immunoassays (“DIA”)”, in T. C. J. Gribnau, J. Visser and R. J. F. Nivard (Eds.), Affinity Chromatograph and Related Techniques. Elsevier, Amsterdam, 411 (1982), and T. Gribnau, A. van Sommeren and F. van Dinther, “DIA—Disperse Dye Immunoassay”, in I. M. Chaiken, M. Wilchek and I. Parikh (Eds.), Affinity Chromatography and Biological Recognition, Academic Press, Orlando, Fla., 375 (1983)). The dispersed dye immunoassay (DIA) described in U.S. Pat. No. 4,373,932 (now in the public domain) involves the use of water-dispersible hydrophobic dye particles, which are coated with antibodies, as labels in a heterogeneous sandwich immunoassay. Such dyes can be drawn from a wide variety of water-dispersible classes, including disperse dyes, transfer dyes, fat dyes (solvent dyes), vat dyes, organic pigments, sulfuric dyes, mordant dyes, solubilized (leuco) vat dyes, solubilized (leuco) sulphur dyes, azoic dyes, oxidation bases and ingrain dyes. Various methods can be used to successfully bind antibodies to the surface of the dye particles without reducing their effective immunochemical activity.

Most importantly, as taught by U.S. Pat. No. 4,373,932, solubilization of the dye particles in an appropriate buffer (e.g. an organic solvent) dramatically intensifies the measured absorbance. This prior art, however, does not disclose a method of conducting an assay in which the fluorescence of solubilized dye is measured. The solubilization of dye is critical, because when in colloidal form, the close proximity of dye molecules leads to rapid non-radiative decay. This process severely quenches the fluorescence of any excited dye molecules and dramatically lowers the fluorescent signal. Conversely, the solubilization of dye particles into a dye solution physically separates adjacent molecules and enables efficient and strong fluorescence. Furthermore, the pH of the dye solution must be accurately controlled in order to enable efficient fluorescence. The enhanced absorption and efficiency of fluorescence of dye in a solubilized form therefore leads to a large enhancement of the fluorescence signal and thus the sensitivity of the assay.

Despite this potential for a superior immunoassay based on the solubilization of bound dye particles, the use of colloidal dyes in immunoassays has been primarily restricted to lateral flow assays. Such assays, also known as dipstick assays, are highly useful in field applications where spectrophotometric equipment, refrigeration and trained personnel are not available. Such assays were initially described by Snowden and Hommel (K. Snowden and M. Hommel, “Antigen Detection Immunoassay Using Dipsticks and Colloidal Dyes”, J. Immunol. Methods 140, 57 (1991)), using a procedure known as the dipstick colloidal dye immunoassay. Instead of solubilizing bound dye particles and measuring absorbance as proposed by Gribnau and coworkers, the dipstick colloidal dye immunoassay uses a nitrocellulose membrane coated with antibodies that is exposed to analyte in the sample. The membrane is then incubated in a colloidal solution of dye particles coated with antibodies, which are bound by the analyte. After washing the membrane in water, the unbound dye particles are removed and the presence of bound dye particles causes a visible colour change. Although such assays are ideal for field applications, they do not answer the need of clinical settings that require a quantitative and sensitive assay.

Therefore, the use of colloidal dyes in immunoassays has to date been rather limited and considerable opportunities remain for their usage in highly sensitive immunoassays with amplification. As disclosed in this invention, an improvement over the prior art method, involving measuring fluorescence from solubilized dye and controlling the dye particle radius, leads to a dramatic improvement in the sensitivity, dynamic range, detection limit, selectivity and accuracy of the assay.

### SUMMARY OF THE INVENTION

Accordingly, the present invention provides a method of conducting an assay for the detection of a target analyte with enhanced sensitivity, dynamic range, detection limit, selectivity and accuracy using a sandwich assay format. A liquid sample is first brought into contact with a solid phase, where the solid phase is coated with receptors that have a high affinity for an analyte that may be present in the sample. After an incubation period in which the analyte binds to the receptors, and is thereby immobilized onto the solid phase, a colloidal solution of dye particles is introduced. The dye particles are coated with a second type of receptor that also has a high affinity for the analyte, but a low affinity for the first receptor and also a low affinity for the solid phase. The dye particles therefore bind to the analyte and become immobilized onto the solid phase. The solid phase is then separated from the liquid phase, which in turn separates the bound dye particles from the unbound dye particles. A solubilization buffer, maintained at an appropriate pH, is then added to solubilize the bound dye particles, creating a dye solution. The fluorescence of the resulting dye solution is measured, wherein the solubilized dye molecules strongly absorb excitation light and emit light with high efficiency, and the concentration of the analyte is determined using a pre-determined standard curve. Thus, the present invention provides an assay for a target analyte comprising the steps of:

a) contacting a solid-phase coated with first receptors having a high affinity for the target analyte with a known sample volume so that any target analyte present in said sample volume binds with said first receptors so that said target analyte is bound to said solid phase;

b) adding a solution containing colloidal dye particles coated with second receptors having high affinity for the target analyte, but low affinity for the solid-phase and the first receptors, so that said coated colloidal dye particles bind to any of the immobilized target analyte present forming bound coated colloidal dye particle-target analyte complexes on the solid-phase;

c) separating said coated colloidal dye particles not bound to said solid phase from the bound coated colloidal dye particle-target analyte complexes on the solid-phase;

d) forming a dye solution by solubilizing dye particles of the bound coated colloidal dye particle-target analyte complexes into a solubilization buffer which is maintained in a pre-selected pH range;

e) measuring fluorescence upon optically exciting said dye solution with excitation light at an appropriate wavelength; and

f) relating said measured fluorescence to a concentration of said target analyte in said known sample volume using a pre-established standard curve.

The present invention also provides a method of conducting an assay for the detection of a target analyte using a competitive assay format, permitting the measurement of analytes with low molecular weights. A liquid sample is first brought into contact with a solid phase, where the solid phase is coated with receptors that have a high affinity for an analyte that may be present in the sample. Immediately after introducing the liquid sample, a colloidal solution of dye particles is also introduced. The dye particles are coated with the target analyte. The dye particles therefore compete with the analyte for the available binding sites of the immobilized receptors on the solid phase. The solid phase is then separated from the liquid phase, which in turn separates the bound dye particles from the unbound dye particles. A solubilization buffer, maintained at an appropriate pH, is then added to solubilize the bound dye particles, creating a dye solution. The fluorescence of the resulting dye solution is measured, wherein the solubilized dye molecules strongly absorb excitation light and emit light with high efficiency, and the concentration of the analyte is determined using a pre-determined standard curve.

Thus, in another aspect of the invention there is provided an assay for a target analyte, comprising:

a) contacting a solid-phase coated with first receptors having a high affinity for the target analyte with a known sample volume so that any target analyte present in said sample volume binds with said first receptors so that said target analyte is bound to said solid phase;

b) adding a colloidal solution containing colloidal dye particles coated with the target analyte, so that the colloidal dye particles compete for binding sites of the immobilized receptors on the solid phase;

c) separating said coated colloidal dye particles not bound to said solid phase from the bound coated colloidal dye particle-target analyte complexes on the solid-phase;

d) forming a dye solution by solubilizing the dye particles not bound to said solid phase into a solubilization buffer which is maintained in a pre-selected pH range;

e) measuring fluorescence upon optically exciting said dye solution with excitation light at an appropriate wavelength;

f) relating said measured fluorescence to a concentration of said target analyte in said known sample volume using a pre-established standard curve.

In a preferred embodiment of the invention, the radius of the dye particles is chosen to lie within a narrow range in order to optimize the sensitivity of the assay via amplification and to optimize the dynamic range of the assay via the control over washing and binding forces.

In another preferred embodiment of the invention, the receptors are immobilized on the surface of magnetic beads. The use of magnetic beads allows for optimization of assay parameters and can be utilized to a) compensate for variations in dye properties, and b) increase receptor surface area and thereby improve signal to noise. The use of receptor-coupled magnetic beads also allows for easier washing and separation steps.

In another preferred embodiment of the invention, multiple assays are multiplexed using different dye colours. In this embodiment, multiple mobile solid supports, or regions on a single solid support, are prepared with surface chemistries having specific affinities for the different analytes. Each distinct surface captures a distinct analyte in the sample with high affinity. Dye particles of multiple colours, each colour having a distinct analyte-specific surface chemistry, are bound by their respective immobilized target analytes. The radius of each type of dye particle can be varied in order to obtain an optimized sensitivity for each individual assay. After solubilization of the captured or unbound dye, the concentration of each dye and hence each analyte is obtained through spectral analysis of the fluorescent signal.

The present invention also provides a method for the detection of a target analyte, comprising the steps of:

a) contacting a solid-phase coated with receptors having a high affinity for the target analyte with a known volume of a liquid sample being tested for a presence or absence of the target analyte, the liquid sample containing a known amount of colloidal dye particles having the target analyte bound thereto, wherein in the absence of target analytes in the liquid sample target analytes bound to the colloidal dye particles bind to the receptors to form colloidal dye particle-target analyte-receptor complex, and in the presence of target analytes in the liquid sample the target analytes preferentially bind to the receptors to form target analyte-receptor complexes;

b) removing the solid phase from contact with said liquid sample and forming a dye solution by exposing the solid phase to a solubilizing solvent for solubilizing any dye particles of the colloidal dye particle-target analyte-receptor complexes into a solubilization buffer;

c) measuring fluorescence upon optically exciting said dye solution with excitation light at an appropriate wavelength; and

d) relating said measured fluorescence to a concentration of said target analyte in said known sample volume using a pre-established standard curve.

The present invention also provides a method for the detection of a target analyte, comprising the steps of:

a) contacting a first solid-phase coated with receptors having a high affinity for the target analyte with a known volume of a liquid sample being tested for a presence or absence of the target analyte, the liquid sample containing a known amount of colloidal dye particles having the target analyte bound thereto, wherein in the absence of target analytes in the liquid sample target analytes bound to the colloidal dye particles bind to the receptors to form colloidal dye particle-target analyte-receptor complex, and in the presence of target analytes in the liquid sample the target analytes preferentially bind to the receptors to form target analyte-receptor complexes;

b) separating said coated colloidal dye particles not bound to said solid phase from the bound coated colloidal dye particle-target analyte complexes on the solid-phase;

c) forming a dye solution by solubilizing the dye particles not bound to said solid phase into a solubilization buffer which is maintained in a pre-selected pH range;

d) measuring fluorescence upon optically exciting said dye solution with excitation light at an appropriate wavelength; and

e) relating said measured fluorescence to a concentration of said target analyte in said known sample volume using a pre-established standard curve.

A further understanding of the functional and advantageous aspects of the invention can be realized by reference to the following detailed description and drawings.

### BRIEF DESCRIPTION OF THE DRAWINGS

The invention will now be described, by way of non-limiting examples only, reference being had to the accompanying drawings, in which:

FIG. 1 is a flow chart illustrating the steps involved in the assay if a solid phase was used.

FIG. 2 shows the process steps in a competitive assay where the receptors have been immobilized on magnetic beads.

FIG. 3 shows the dose-response curve of a competitive assay for morphine where the dependence of the fluorescence of the bound dye on the analyte concentration is plotted.

FIG. 4 illustrates the response of a competitive assay for morphine where the dependence of the fluorescence of the unbound dye on the analyte concentration is plotted.

FIG. 5 is a schematic of an optical detection cell optimized for absorbance measurements with a long path length.

FIG. 6 plots an example of the probability distributions of the contact and washing forces, p_{c}(F) and p_{w}(F).

FIG. 7 plots an example of the probability distributions of the net binding force for dye particles bound by one, two and three bonds.

FIG. 8 plots the number of solubilized dye molecules as a function of the number of analyte molecules bound on to the solid support for different dye particle radii in a simulated assay, demonstrating the existence of an optimal dye particle radius.

FIG. 9 plots the number of solubilized dye molecules as a function of the number of analyte molecules bound on to the solid support for different dye particle radii, demonstrating the sensitivity of the assay to cross-talk phenomena.

FIG. 10 plots the number of solubilized dye molecules as a function of the number of analyte molecules bound on to the solid support for different dye particle radii, demonstrating the sensitivity of the assay to variations in the binding force.

### DETAILED DESCRIPTION OF THE INVENTION

The present invention provides a novel immunoassay for the detection of analytes using dye particles. More specifically, the present invention describes a method of performing a heterogeneous or homogeneous binding assay using dye particles as chromatic labels that are detected via absorbance or fluorescence following their solubilization.

The term “receptor”, as used herein, means antibodies, antigens, DNA, RNA, nucleic acids, aptamers, enzymes, or any other molecular species capable of exhibiting a specific binding affinity for the analyte.

The term “analyte”, as used herein, means antibodies, antigens, nucleic acids, aptamers, enzymes, molecules, proteins, viruses, bacteria, ions, or any species whose presence or concentration in a sample is sought.

The term “solid support”, as used herein, means a surface onto which the first receptor molecules can be coated, adsorbed or bound. For example, the solid support may be a microwell, the walls of a capillary tube, or a microsphere.

The term “colloidal dye”, as used herein, means disperse dyes, transfer dyes, fat dyes (solvent dyes), vat dyes, organic pigments, sulfuric dyes, mordant dyes, solubilized (leuco) vat dyes, solubilized (leuco) sulphur dyes, azoic dyes, anthraquinine dyes, coumarin dyes, oxidation bases and ingrain dyes.

The term “solubilization buffer”, as used herein, means any buffer capable of entirely or nearly entirely solubilizing the dye particles, e.g. an alkaline solution or organic solvent.

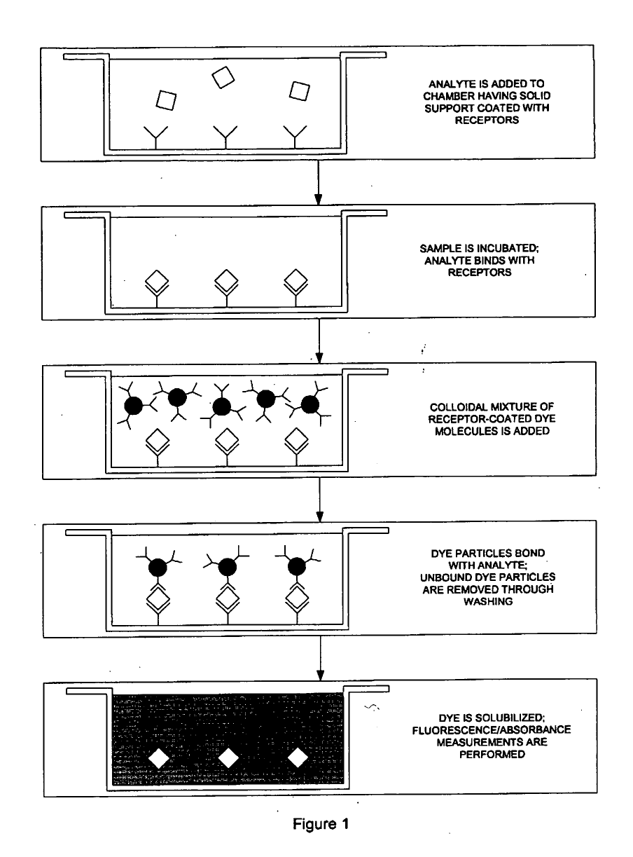

FIG. 1 shows a flow chart describing a two-site sandwich immunoassay representative of an embodiment of the present invention. A solid support is coated with a receptor having a high affinity for the analyte under consideration. The sample is introduced and analyte molecules are bound to the solid support with one or more receptor molecules via specific chemical bonding following a brief incubation period. A colloidal solution of dye particles coated with a second type of receptor (also having a high affinity for the analyte), is then introduced. During incubation, the dye particles bind to the analyte via the attached receptor molecules and are thus immobilized on the solid support. The unbound dye particles are washed using a liquid washing buffer, leaving only the dye particles bound by the analyte. A solubilization buffer is added and the bound dye particles are solubilized, releasing their colour into solution. The concentration of the dye is measured via a fluorometric measurement and the analyte concentration is obtained using a pre-established standard curve relating the measured signal to the analyte concentration.

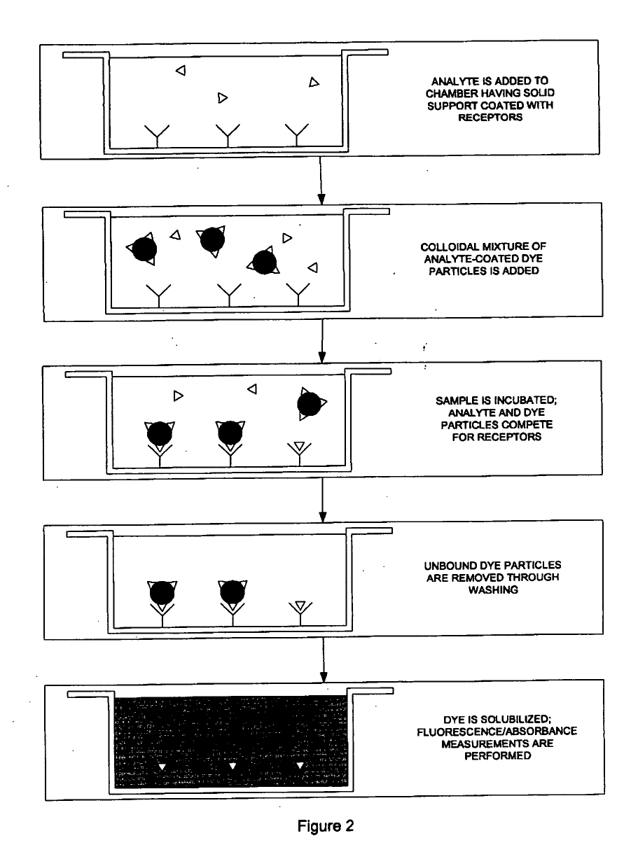

An alternative assay format is shown in FIG. 2, where a flow chart of a competitive assay is described. As in FIG. 1, a solid support is coated with a receptor having a high affinity for the target analyte. The sample is introduced and analyte molecules are bound to the solid support via specific chemical bonding. A colloidal solution of dye particles coated with the analyte is then introduced. During incubation, the analyte-coated dye particles compete with the analyte from the sample volume for binding sites of the receptors on the solid phase. The unbound dye particles are washed using a liquid washing buffer, leaving only the dye particles bound by the analyte. A solubilization buffer is added and the bound dye particles are solubilized, releasing their colour into solution. The concentration of the dye is measured via a fluorometric measurement and the analyte concentration is obtained using a pre-established standard curve relating the measured signal to the analyte concentration.

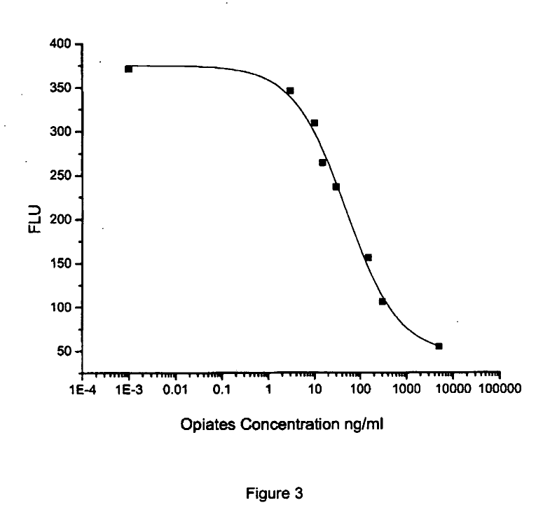

FIG. 3 shows an exemplary dose-response curve of the assay in competitive format where the target analyte is morphine. As can be appreciated from this graph, the signal contrast is more than a factor of ten in arbitrary fluorescing units.

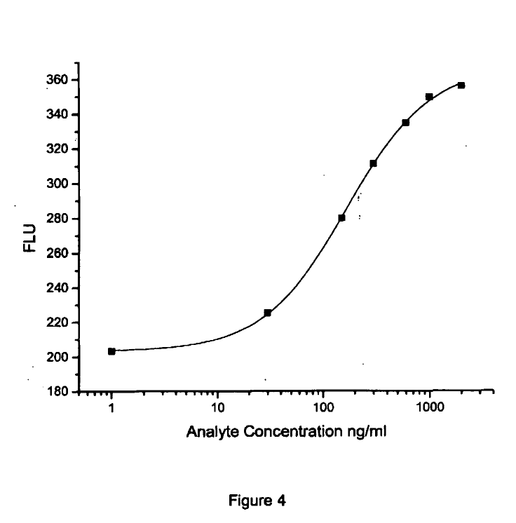

Instead of following the process steps as highlighted in FIGS. 1 and 2 in which the analyte concentration is deduced from the fluorescence of solubilized bound dye, it is possible to infer the analyte concentration from a measurement of the unbound dye. This is achieved by solubilizing the unbound dye particles after the separation step and measuring the fluorescence of the unbound solubilized dye. This approach has the added benefit of eliminating the wash steps that can prolong the assay time. The major drawback of this assay format is its limited dynamic range relative to the assay format in which the bound dye is measured. FIG. 4 shows an exemplary dose-response curve of this assay format, in which the target analyte is morphine.

The method of amplification via solubilization disclosed in U.S. Pat. No. 4,373,932 was described primarily in the context of increasing the signal produced by absorbance of the bound dye. In particular, measurements of the increase in absorbance before and after solubilization were provided, demonstrating this effect. However, the process of solubilization can lead to even greater benefits for assays based on fluorometric measurements, and the prior art fails to teach a method to realize this benefit. In addition to the amplification of the fluorescence signal due to a better penetration of excitation light and more homogeneous excitation of the dye molecules, the quantum yield will be markedly improved as a result of inhibited quenching. When an excited dye molecule is in colloidal form, the close proximity of other molecules allows the non-radiative transfer of energy in a process known as self-quenching. This process can cause a large reduction in the quantum yield, significantly reducing the sensitivity and accuracy of an assay. However, upon solubilization, molecules can be efficiently excited and lack the self-quenching decay channel, allowing for a very high quantum yield. It is also noted that both fluorescence and absorbance measurements may be carried out using a cell that is optimally designed for high absorption and the efficient excitation and collection of fluorescence, in order to provide a potentially more sensitive measurement.

In a preferred embodiment of the invention, magnetic beads are used as a mobile solid phase for the separation and extraction of bound dye particles. The separation of the magnetic beads from the surface is performed using one of many known methods in the prior art, all of which use a magnetic field to spatially isolate the magnetic beads. Magnetic beads enable a significant enhancement in the repeatability and ultimately the precision of the assay. This enhancement is possible because the number of magnetic beads in the assay, and hence the amount of surface area for immobilizing dye particles, can be accurately controlled. This is particularly important for assays employing colloidal dye particles, since variations can exist in the affinity of bound receptors, and the smoothness, size and geometry of the dye particles. Therefore, the control over surface area provided by the number of magnetic beads used in an assay offers a means of accurately compensating for batch-to-batch variations the properties of dye particles. Furthermore, magnetic beads, by their very nature as a mobile solid phase evenly distributed within a liquid phase, allows for a more uniform reaction between the dye particles, analyte, and receptors, which reduces the time required for the assay incubation. Finally, the use of magnetic beads allows for many convenient and easily automated methods of extraction that are known in the prior art.

The colloidal solution can be prepared using the methods taught in U.S. Pat. No. 4,373,932 by Gribnau et al., in which numerous techniques are disclosed for the preparation of dye sols coated with antibodies, which patent is incorporated herein in its entirety. These methods can be generalized to the preparation of dye particles coated with other receptor capture agents, including aptamers. A preservative such as thimersol can be added to the colloidal solution to provide a long and stable shelf life.

In the prior art, the means of optical detection of the solubilized dye has focused almost exclusively on colourimetry. Although the absorbance of the dye can indeed be amplified by solubilization, the degree of amplification depends critically on the geometry of the optical cell used for absorbance. Unfortunately, no consideration of this important element of the assay design has been given in the prior art. A dramatic enhancement in the degree of amplification can be obtained by a careful choice of the optical cell used to measure the absorbance of the solubilized dye. In particular, transferring the solubilized dye solution into a long and narrow capillary cell allows for a large increase in the optical path length of an absorbance measurement. Furthermore, the use of a low index cladding material, such as Teflon, offers the ability to provide a liquid waveguide for the optical beam used in an absorbance measurement, with path lengths limited only by the volume of the dye solution.

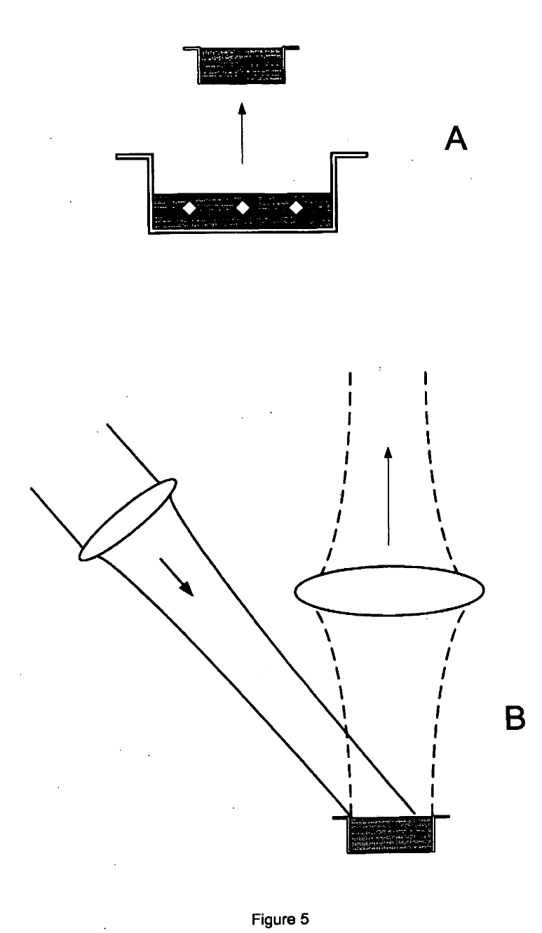

FIG. 5 shows an example illustrating the concept of amplification through solubilization and path length enhancement. A small volume of solubilization buffer is chosen to wet the solid surface upon which dye particles are bound during incubation. The dye solution formed following solubilization is placed in a capillary tube, either by pipetting, centrifugal force or capillary action, and the dye solution fills the capillary. The capillary may be a single capillary tube or a capillary housed in a cartridge containing single or multiple capillaries. An optical beam is directed along the axis of the capillary tube and propagates through the capillary tube before encountering the optical detector.

The optical beam may be produced either by a laser or an incoherent source such as a light emitting diode or lamp. If the beam is sufficiently collimated that it does not encounter the walls of the capillary tube during propagation, the capillary tube need not be a waveguide with Teflon cladding. If a monochromatic or narrow-spectrum source is used, no filtering element is needed prior to detection. However, if a broadband source is used, a filtering element placed either before or after the measurement cell is required. In a preferred embodiment, a polychromatic light source is used in order provide one measurement of the transmission through the cell within the bandwidth of the dye's absorbance, and another measurement of the transmission through the cell outside of the dye's absorbance bandwidth. This second measurement facilitates the subtraction of background broadband losses due to poor coupling and scattering. Finally, if multiple assays are multiplexed in the same incubation chamber, with a different coloured dye particle for each assay, additional beams or filtering elements can be added in order to spectrally resolve and quantify the absorbance of each dye. If a detailed measurement of the absorbance spectrum is obtained (i.e. absorbance measurements at multiple spectral points), curve fitting methods can be used to extract the individual contributions of different dye particles with partially overlapping spectra, increasing the number of dyes (assays) that can be multiplexed and also increasing the sensitivity of each individual measurement.

Although the sensitivity of the assay can be significantly improved by controlling the geometry of the optical measurement cell and using the fluorescence of solubilized dye to avoid self-quenching, it can be further improved by controlling the dye particle radius to obtain optimal specific binding and minimal non-specific binding following a washing step. The sensitivity of a binding assay is highly dependent on the detailed chemical nature and forces present during the binding process. For example, in a sandwich assay employing single-molecule dye labeling (rather than a large dye particle), it is often very difficult to remove dye-labeled receptor molecules that bond non-specifically to the surface. If such molecules cannot be removed, the sensitivity of the assay will be degraded by a large background signal. Although the term “washing” is commonly applied to the process of removing unbound and non-specifically-bound dye-labeled receptor molecules, the washing process in this case is not characterized by an applied fluidic force. Indeed, due to the presence of the boundary layer, a moving fluid is unable to effectively remove non-specifically bound molecules. Instead, the washing process uses a probabilistic thermal escape process to induce the release and removal of non-specifically bound molecules. The probability of escape of a bound molecule with a binding energy of E_{b} at a temperature T is proportional to e^{-Eb/kT}, where k is Boltzman's constant. If the binding energy is not too much larger than kT (26 meV at room temperature), then there can be a high probability that the molecule will escape over a given time interval. A certain percentage of non-specifically bound dye particles can then escape and be removed simply by incubating the solution at a given temperature. Although the binding energy of a typical specific bond is on the order of 0.3-0.8 eV, the binding energy of a non-specific bond can take on a wide range of values, depending on the affinity of the interaction. It follows that this process is very inefficient and will inevitably lead to inefficient washing and a significant background signal from unwashed non-specifically bound dye-labeled molecules. In contrast to the single-molecule washing method, washing forces can be applied to larger microscopic particles. Therefore, the assay embodied by the present invention offers the potential to optimize these forces for the efficient removal of non-specifically bound dye particles without disturbing the specifically bound particles. Unlike other assays utilizing the optical identification and enumeration of large micron-sized microspheres that require that the particle size be sufficiently large for optical resolution in a microscope, the assay of the present invention provides the flexibility to optimize over a very wide range of nanoscopic to microscopic particle sizes.

In order to quantify this concept further, it is useful to consider a simple model of the forces involved during the specific and non-specific binding of a dye particle in the inventive assay. We consider first a solid support with an area of Aₛ that is uniformly coated with receptor molecules. Given a spherical dye particle with radius R, the effective contact area between the dye particle and the surface can be approximated by
A_{cell}=0.181R (1)
(see K. Cooper et al., “Simulation of the Adhesion of Particles to Surfaces”, J. Colloid. Interface Sciences 234, 284 (2001)), where R is in units of μm and A_{cell} is in units of μm². The number of individual contact area elements of area A_{cell} on the entire solid support area Aₛ is given by

$${N}_{cell} = \frac{{A}_{s}}{{A}_{cell}} = \frac{5.53 {A}_{s}}{R} . \qquad ( 2 )$$

Following the incubation of a sample containing a given concentration of analyte molecules, we assume that Nₐ analyte molecules bind to the surface via attachment to receptor molecules so that the average number of analyte molecules per contact area element is

$$μ = \frac{{N}_{a}}{{N}_{cell}} . \qquad ( 3 )$$

For a given particle radius, the parameter μ is linearly proportional to the concentration of analyte molecules in the sample volume, and is used henceforth as a measure of analyte concentration.

Following the incubation of dye particles coated with receptor molecules, a total of N_{b} dye particles specifically bind to the surface. A further N_{c} dye particles bind non-specifically to the surface through frictional contact forces. Since a single dye particle can be bound by more than one analyte-receptor bond, it follows that N_{b}<Nₐ, and the number of bound dye particles must be calculated using statistical methods. Although the average number of analyte particles per contact area element is μ, the value of μ will typically be much less than unity. One must therefore calculate N_{b} by considering the probability distribution of μ, which can be shown to be a Poissonian distribution:

$${p}_{k} = \frac{{μ}^{k} {ⅇ}^{- μ}}{k !} , \qquad ( 4 )$$

where pₖ is the probability of that a given contact area element will have k analyte molecules, and therefore k bonds to a single dye particle. This relation allows one to calculate N_{bk}, the number of dye particles that will be bound with k bonds, by writing N_{bk}=pₖN_{cell}, with the total number of bound dye particles given by

$${N}_{b} = {N}_{cell} {\sum }_{k = 1}^{∞} {p}_{k} . \qquad ( 5 )$$

It is important to note that in the limit of a large number of dye particles binding, the surface coverage will be saturated and the above expression will no longer be valid. The surface coverage is assumed to be saturated when N_{b}˜Nₛₐₜ/2, where

$${N}_{sat} = \frac{{A}_{s}}{{A}_{sphere}} = \frac{{A}_{s}}{π {R}^{2}} .$$

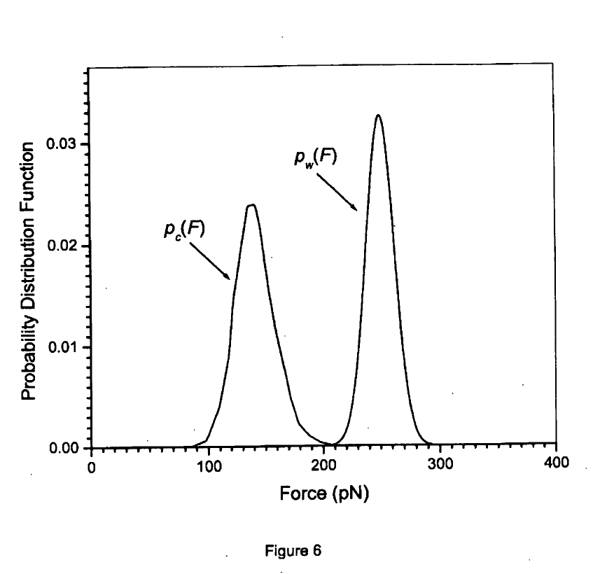

Although equation (5) provides an expression for the number of bound dye particles, the model is incomplete because it has not yet considered the effect of washing on the number of bound particles. In order to do so, one must consider the forces acting on a dye particle during the washing process. These forces include the binding force due to the analyte-receptor bonds F_{b}, the contact force between a dye particle and the solid support due to frictional (van der Waals) forces F_{c}, and the washing force F_{w}. Reported values for F_{b} have ranged from low tens of pN to approximately 250 pN, depending on the affinity of the analyte-receptor interaction. However, the contact force is by definition statistical in nature, since small variations in the surface roughness of the dye particle or solid support can lead to large differences in the contact force. It is therefore appropriate to consider the contact force as a force probability density function p_{c}(F), which peaks at the average contact force F_{c}. A similar argument can be applied to the washing force (the applied force), which can vary due to geometrical effects, turbulence and orientational effects, and is described by a second probability density function p_{w}(F) that peaks at F_{w}. FIG. 6 shows an example of the relationship between the probability density functions p_{c}(F) and p_{w}(F). In this example, the non-specifically bound particles (bound via the contact force) are efficiently washed due to the fact that F_{w}>F_{c}.

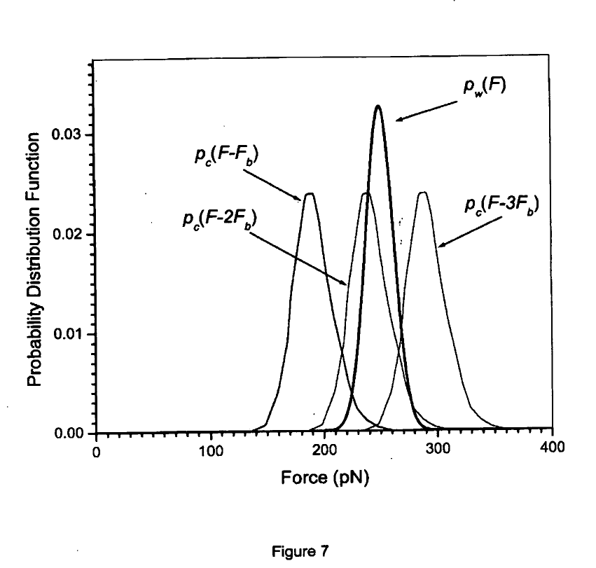

If the binding force F_{b} is small relative to F_{w}, then dye particles bound by one or two analyte-receptor bonds will also be washed away, decreasing the sensitivity of the assay. This will decrease the value of N_{b} relative to that obtained in equation (5). The effect of the washing force on dye particles with multiple analyte-receptor bonds is illustrated in FIG. 7 (assuming a binding force of 50 pN). The net force binding a dye particle is given as the sum of the contact force and k times the binding force. Since the contact force is described by a probability distribution function, the net binding force for a dye particle bound by one bond (and the contact force) is given by the probability distribution p_{c}(F-F_{b}). Similarly, the binding force for a dye particle bound by k bonds (and the contact force) is given by the probability distribution p_{c}(F-kF_{b}). A given dye particle will only remain bound after washing if some or all of p_{c}(F-kF_{b}) lies beyond p_{w}(F). In the case of FIG. 7, it is clear that most of the dye particles bound by single and double bonds will be removed by washing. The probability \tilde{p}ₖ that a given bead with k bond will remain intact after washing can be calculated as follows:

$${\tilde{p}}_{k} = {\int }_{0}^{∞} {p}_{c} ( F - {kF}_{b} ) {\int }_{0}^{F} {p}_{w} ( {F}^{′} ) ⅆ {F}^{′} ⅆ F . \qquad ( 6 )$$

Having considered the effect of washing forces, it is now possible to calculate the total number of bound beads N_{b}′ that remain after washing. This is done by rewriting equation (5) and including the probability \tilde{p}ₖ from equation (6):

$${N}_{b}^{′} = {N}_{cell} {\sum }_{k = 1}^{∞} {p}_{k} ( μ ) {\tilde{p}}_{k} ( {p}_{c} , {p}_{w} , {F}_{b} ) .$$

This expression clearly establishes the link between the measured optical signal from the dye (which is proportional to N_{b}′) and the analyte concentration μ and force parameters p_{c}, p_{w} and F_{b}.

The above model provides a quantitative relationship between the sensitivity of the assay and the relevant physical parameters. However, the essential observation to be made is that many of the parameters depend critically on the dye particle radius R. These parameters include the number of individual contact area elements N_{cell} α R⁻¹, the contact force F_{c} and its distribution width, which increase with R, and the washing force F_{w} and its distribution width, which also increase with R. Finally, the optical signal generated via absorbance or fluorescence is proportional to N_{b}′, which is itself proportional to R³.

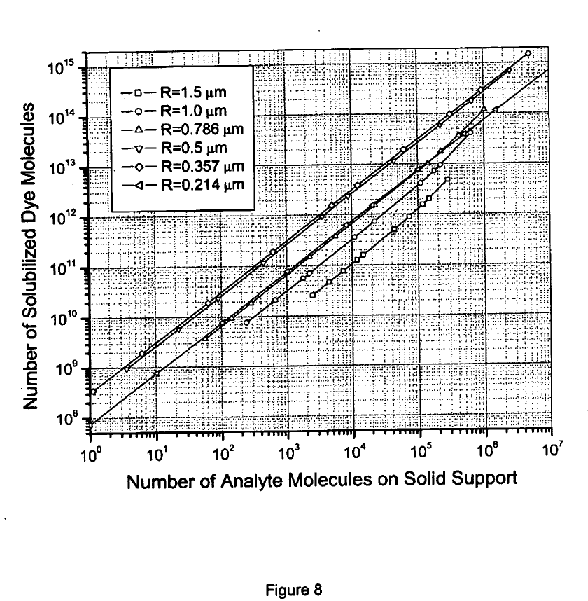

The various dependencies of the assay parameters on R clearly indicate that a trade off will exist between efficient washing and having a large number of bound particles (low R regime) and amplification via solubilization (high R regime). This fact is illustrated in FIG. 8 for a simulated assay with efficient washing and a binding force of 50 pN and a solid support area of Aₛ=4 mm². In this figure, the number of solubilized dye molecules is plotted against number of analyte molecules bound on to the solid support, assuming a molar mass of 331 g/M and a specific gravity of unity for each dye molecule. One readily observes that an intermediate value of R˜400 nm provides optimal sensitivity.

In addition to an enhancement in sensitivity, the optimal assay also provides a vast increase in dynamic range. This is apparent in FIG. 8, where it can be seen that the dynamic range of the non-optimized assay is only approximately two orders of magnitude, while the optimized assay has a dynamic range in excess of six orders of magnitude. This dramatic increase in the dynamic range is produced by two effects. Firstly, the minimal detectable analyte concentration is determined by the analyte concentration where only a single particle is bound prior to solubilization.

In the case of large, non-optimized particles, the requirement of multiple bonds per particle (i.e. many bonds are required to survive the large washing force) severely limits the number of bound particles after washing. However, in an optimized assay with smaller particles, one or very few bonds are required to survive washing and the analyte concentration at which a single bead is bound is many orders of magnitude lower than that of a large particle assay. Secondly, as mentioned above, the maximum analyte concentration is estimated by the concentration where the projected surface area of the bound particles (prior to washing) is equal to half the total support area. Clearly, the number of bound particles will be inversely proportional to the particle radius. Indeed, for very large particles far from the optimal radius, the maximum number of analyte particles (i.e. the analyte concentration) is much lower than that of the optimized radius.

Furthermore, since a large percentage of the bound particles of the non-optimized assay are removed by washing (since many bonds are needed to survive the large washing force), the number of solubilized molecules is very low. The optimized assay, however, allows many more particles to bond to the surface at saturation. Since the washing force is sufficiently small to cause minimal removal of bound particles, the number of dye molecules after solubilization is very high. However, it is important to note that if the particles are too small, then the amplification will be very low and the washing force will be too small to eliminate non-specifically bound particles. It is therefore apparent that the optimal assay, with an intermediate radius, provides both high sensitivity and large dynamic range.

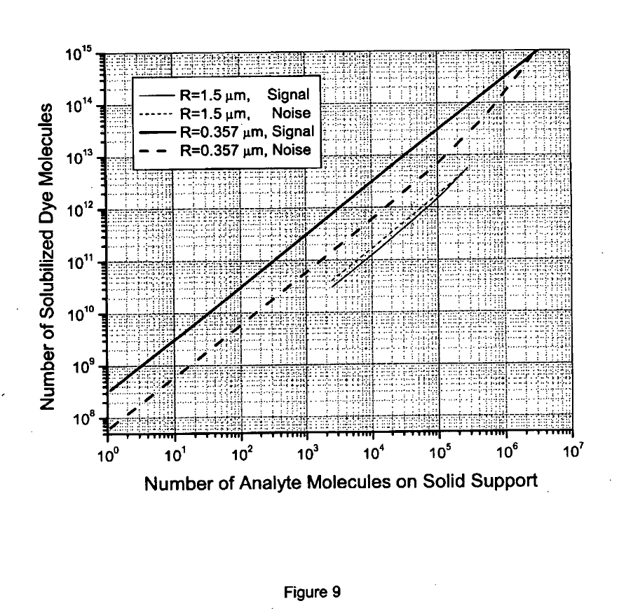

Although the preceding discussion demonstrates that the sensitivity and dynamic range of the dye solubilization assay may be optimized by controlling the particle radius, it can also be shown that this optimization procedure leads to enhanced specificity. The specificity is determined by the sensitivity of the assay to non-specific binding events. These events, in most cases, will have binding forces significantly lower than the primary specific analyte bond. However, if the washing process is inefficient, some of these weaker bonds may remain, causing the assay noise floor to rise. However, if the particle size is optimized so that the washing force and contact force are of similar magnitude to the binding force, then a situation can occur in which a single specific bond will not be broken by washing, while there is a high probability that a weaker non-specific bond will be broken. In such a case, the washing process improves the specificity of the assay. The effect of “specific washing” is demonstrated in FIG. 9, where the number of solubilized dye molecules is plotted as a function of the number of analyte molecules bound on to the solid support for two different radii—one near the optimization point (R˜357 nm) and one much larger (R˜1500 nm). An analyte with a binding force of 50 pN is simulated and a second cross-reacting species with a binding force of 20 pN is also assumed to be present. The signal due to the additional cross-reacting species is indicated on the figure as “noise”, and the concentration of the cross-reacting species is assumed to be 100 times that of the analyte at a given analyte concentration. As clearly shown in the figure, the noise exceeds the signal for the non-optimized assay. However, for the optimized assay, the noise signal is always almost an order of magnitude less than the signal. This illustrates that optimization provides the additional benefit of the lowest background due to non-specific binding events.

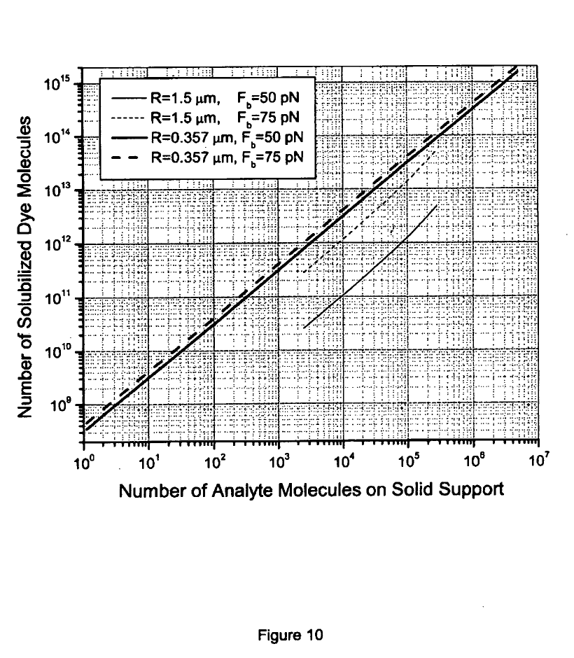

An additional benefit beyond sensitivity, dynamic range and specificity is insensitivity to variations in affinity. If the assay is optimized in such a way that the binding force is of similar magnitude to the washing and contact forces, then, as describe above, a single bond can survive the washing step. In this case, any additional affinity (bond strength) will have a negligible effect on the number of bound particles after washing. However, if the assay is not optimized and multiple bonds are required, the assay will be very sensitive to subtle changes in affinity. Such affinity variations are often present when antibodies are used as receptors in an immunoassay. This principle is illustrated in FIG. 10, where number of solubilized dye molecules is again plotted as a function of the number of analyte molecules bound on to the solid support for two different radii (optimized and non-optimized). The non-optimized assay is very sensitive to the binding force, with an increase in the binding force of only 25 pN producing a change of an order of magnitude in the number of solubilized dye particles. In contrast, the number of solubilized dye molecules in the optimized assay is nearly independent of the increase in binding force. Therefore, the optimized assay provides the additional benefit of insensitivity to variations in analyte-receptor bond affinity.

The present invention, describing improvements to the dispersed dye immunoassay, incorporates this optimization step into the design of the assay. This optimization process may be conducted empirically by determining the dependence of sensitivity and dynamic range on particle radius. Since the binding force for different analytes will vary in strength, the radius of the dye particle should be optimized uniquely for each analyte. This enables the design of a multiplexed assay (using different colours) for several different analytes, with each individual assay having an optimized sensitivity and dynamic range.

Finally, as previously mentioned, it should be apparent to those skilled in art that the receptor molecules attached to the dye particles can include nucleic acid oligonucleotides for the detection of DNA or RNA via a hybridization reaction. Furthermore, the receptor molecules attached to the solid support in a sandwich assay may also be oligonucleotides, facilitating the formation of a DNA sandwich assay. Apatmers, which are also formed out of nucleic acids, may be used for the detection of a wide range of antigens.

As an example, the coumarin family of disperse textile dyes provides an excellent chemistry for the attachment of nucleic acid receptors. In particular, the commerically available dye Luminous Red G possesses unique surface chemical and structural characteristics that could allow it to be efficiently used as a hetero-functional solid support with mild surface modification or no modification at all. Indeed, this dye has functional groups capable of reacting with two or more chemically distinct functional linkers, e.g. amines, thiols and carboxy groups. The linkers could serve two purposes: to covalently bind two distinct chemical entities which otherwise would remain non-reactive toward each other and as a physical spacer that provides greater accessibility and freedom to each of the linked bio-molecules such as thiol-modified DNA oligomers or amino-modified oligos. In addition, as a result of the reactive nature of the dye hetero-functional groups, the covalent linkage to bio-molecules is highly stable, eliminating the possibility of leakage from the dye surface. Such resilience leads to enhanced sensitivity and dynamic range of the assay.

The immobilization of oligo-receptor molecules onto the surface of the dye particle can be performed using either covalent or non-covalent bonding. An example of the steps involved in covalent bonding is provided below, in which a 5′ amino linker covalently binds to a disperse dye:

1. Wash 100 mg disperse dye beads 3 times for 5 minutes each, in reagent grade water with centrifugation.

2. Air dry the beads for 10 minutes and add 1 ml of 200 g/L EDAC (1-ethyl-3-(3-dimethylaminopropyl carbodiimide), and mix for 15 min.

3. Rinse 2 times in H₂O and dry at room temperature for 10 minutes.

4. Dilute Oligo (0.5-10 pmol) in Buffer (0.5 M NaHCO₃, pH; 8.4, and 0.1% v/v Tween 20)

5. Add 100 mg of the washed (steps 1-3) dye beads to 1 ml of the solution of step 4.

6. Mix the resulting solution with agitation for 1 hour.

7. Wash the mixture 3 times, each time for 5 min in reagent grade water with centrifugation

8. Add 0.1 N NaOH and agitate for 20 minutes in order to quench remaining active group.

9. Wash 3 times for 5 min. each in reagent grade water with centrifugation and Air dry

10. Store desiccated at 4° C.

The immobilization of oligonucleotides onto a disperse dye can be performed using non-covalent bonding. Examples of non-covalent immobilization are provided in the three examples below:

A) EDC Protocol:

1. Wash the dye beads through the steps 1-3 of the preceding example

2. Add 50 mL of a 10 mM EDC containing 10 pmol of oligo to 50 mg to the washed beads

3. Incubate overnight at around 37° C. with agitation

4. Wash with TNTw sol.

5. Store at 4° C. (can be kept for over period of time)

B) CTAB Protocol

1. Add 50 μL of a 0.03 mM CTAB containing 10 pmol of oligo to washed dye

2. Incubate overnight at around 37° C. with agitation

3. Wash with TNTw sol.

4. Store at 4° C. (can be kept for over period of time)

C) NaCL Protocol

Version I:

1. Add 50 μL of a 0.2 nmole/ml oligo solution in 500 mM NaCL to washed dye

2. Incubate Incubate overnight at around 37° C. with agitation

3. Wash with TNTw sol.

4. Store at 4° C. (can be kept for over period of time)

Version II:

1. Add 50 μL of a 0.2 nmole/ml oligo in 3× PBS (0.15 M phosphate 0.45 M NaCl, pH:7 to washed dye and Incubate 2 hours at 37° C.

2. Wash 3× with 1× PBS containing 0.05% Tween 20 (PBST)

3. Block with 1% skimmed milk or BSA in 1× PBS for 1 hour at 37° C.

4. Store at 4° C. (can be kept for over period of time)

As will be clear to those possessing the ordinary skill of the art, many variations and modifications of the present invention are possible that do not diverge from its scope and spirit. It is therefore to be understood that, within the scope of the preceding disclosure, the invention may be practiced otherwise than as specifically claimed.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES CITED

Patent Documents

1. D. Trau et al., DE10042023 (2003).

2. T. C. J. Gribnau et al., U.S. Pat. No. 4,373,932 (1983).

Other Publications

1. Trau et al., “Nanoencapsulated Microcrystalline Particles for Superamplified Biochemical Assays”, Anal. Chem. 74, 5480 (2002).

2. H. A. Rongen et al., “Liposomes and Immunoassays”, J. Immunol. Methods 204, 105 (1997).

3. A. Kamyshny and S. Magdassi, “Chemiluminescence Immunoassay in Microemulsions”, Colloids Surf. B 11, 249 (1998).

4. T. Gribnau et al., “The Application of Colloidal Dye Particles as Labels in Immunoassays: Disperse(d) Dye Immunoassays (“DIA”)”, in T. C. J. Gribnau, J. Visser and R. J. F. Nivard (Eds.), Affinity Chromatograph and Related Techniques, Elsevier, Amsterdam, 411 (1982).

5. Gribnau, A. van Sommeren and F. van Dinther, “DIA—Disperse Dye Immunoassay”, in I. M. Chaiken, M. Wilchek and I. Parikh (Eds.), Affinity Chromatography and Biological Recognition, Academic Press, Orlando, Fla., 375 (1983).

6. K. Snowden and M. Hommel, “Antigen Detection Immunoassay Using Dipsticks and Colloidal Dyes”, J. Immunol. Methods 140, 57 (1991).

7. K. Cooper et al., “Simulation of the Adhesion of Particles to Surfaces”, J. Colloid. Interface Sciences 234, 284 (2001).

1. A method for the detection of a target analyte, comprising the steps of:
a) contacting a solid-phase coated with first receptors having a high affinity for the target analyte with a known sample volume so that any target analyte present in said sample volume binds with said first receptors so that said target analyte is bound to said solid phase;
b) adding a colloidal solution containing colloidal dye particles coated with second receptors having high affinity for the target analyte, but low affinity for the solid-phase and the first receptors, so that said coated colloidal dye particles bind to any of the immobilized target analyte present forming bound coated colloidal dye particle-target analyte complexes on the solid-phase;
c) separating said coated colloidal dye particles not bound to said solid phase from the bound coated colloidal dye particle-target analyte complexes on the solid-phase;
d) forming a dye solution by solubilizing dye particles of the bound coated colloidal dye particle-target analyte complexes into a solubilization buffer which is maintained in a pre-selected pH range;
e) measuring fluorescence upon optically exciting said dye solution with excitation light at an appropriate wavelength; and
f) relating said measured fluorescence to a concentration of said target analyte in said known sample volume using a pre-established standard curve.

2. The method of claim 1 wherein said sample volume includes additional different types of target analytes, and wherein said solid-phase is coated with additional different types of first receptors having a high affinity for the additional different target analytes so that said additional different types of target analytes bind with said additional different types of receptors so that said additional different types of target analytes are bound to said solid phase, and wherein said colloidal solution contains additional different types of colloidal dye particles coated with additional different types of second receptors having a high affinity for the additional different target analytes but low affinity for the solid-phase and the first receptors and additional different types of first receptors so that said additional different types of target analytes bind with said additional different types of receptors, and wherein forming a dye solution includes solubilizing the additional dye particles, and wherein measuring fluorescence includes exciting each additional dye in the dye solution with excitation light at an appropriate wavelength, and including relating said measured fluorescence to a concentration of each additional different type of target analyte in said known sample volume using pre-established standard curves.

3. The method of claim 1 wherein a radius of the colloidal dye particles have a radius in a pre-selected range.

4. The method of claim 2 wherein a radius of the additional colloidal dye particles have a radius in a pre-selected range.

5. The method of claim 2 wherein the additional types of colloidal dye particles are substantially monodisperse.

6. The method of claim 1 wherein a radius of the colloidal dye particles have a radius in a range from about 10 nm to about 500 nm.

7. The method of claim 1 wherein the colloidal dye particles are substantially monodisperse.

8. The method of claim 1 wherein said solid phase is an interior surface of a liquid sample container, and wherein step c) includes drawing out liquid from said sample container.

9. The method of claim 1 wherein the solid-phase includes a plurality of magnetic particles, said magnetic particles being contained in a vessel, and wherein step c) includes using a magnetic field to enable separation of the coated colloidal dye particles not bound to said solid phase from the bound coated colloidal dye particle-target analyte complexes on the solid-phase.

10. The method of claim 9 including a step of controlling a surface area of the solid-phase by using a pre-selected amount of said magnetic particles.

11. The method of claim 9 including using a pre-selected amount of said magnetic particles for compensating for variations in properties of said dyes, including the affinity of bound receptors, and the smoothness, size and geometry of the dye particles.

12. The method of claim 1 including a step of extending a dynamic range of the assay by measuring an absorbance of said dye solution in addition to the fluorescence of said dye solution, and wherein said measured absorbance and fluorescence are related to a concentration of said target analyte in said known sample volume using pre-established standard curves.

13. The method of claim 1 wherein said step of separating said coated colloidal dye particles not bound to said solid phase from the bound coated colloidal dye particle-target analyte complexes on the solid-phase includes removing said solid phase from the liquid sample and washing said solid phase using a suitable solvent.

14. A method for the detection of a target analyte, comprising the steps of:
a) contacting a solid-phase coated with first receptors having a high affinity for the target analyte with a known sample volume so that any target analyte present in said sample volume binds with said first receptors so that said target analyte is bound to said solid phase;
b) adding a colloidal solution containing colloidal dye particles coated with second receptors having high affinity for the target analyte, but low affinity for the solid-phase and the first receptors, so that said coated colloidal dye particles bind to any of the immobilized target analyte present forming bound coated colloidal dye particle-target analyte complexes on the solid-phase;
c) separating said coated colloidal dye particles not bound to said solid phase from the bound coated colloidal dye particle-target analyte complexes on the solid-phase;
d) forming a dye solution by solubilizing the dye particles not bound to said solid phase into a solubilization buffer which is maintained in a pre-selected pH range;
e) measuring fluorescence upon optically exciting said dye solution with excitation light at an appropriate wavelength;
f) relating said measured fluorescence to a concentration of said target analyte in said known sample volume using a pre-established standard curve.

15. The method of claim 14 wherein said sample volume includes additional different types of target analytes, and wherein said solid-phase is coated with additional different types of first receptors having a high affinity for the additional different target analytes so that said additional different types of target analytes bind with said additional different types of receptors so that said additional different types of target analytes are bound to said solid phase, and wherein said colloidal solution contains additional different types of colloidal dye particles coated with additional different types of second receptors having a high affinity for the additional different target analytes but low affinity for the solid-phase and the first receptors and additional different types of first receptors so that said additional different types of target analytes bind with said additional different types of receptors, and wherein forming a dye solution includes solubilizing the additional dye particles, and wherein measuring fluorescence includes exciting each additional dye in the dye solution with excitation light at an appropriate wavelength, and including relating said measured fluorescence to a concentration of each additional different type of target analyte in said known sample volume using pre-established standard curves.

16. The method of claim 14 wherein a radius of the colloidal dye particles have a radius in a pre-selected range.

17. The method of claim 15 wherein a radius of the additional colloidal dye particles have a radius in a pre-selected range.

18. The method of claim 15 wherein the additional types of colloidal dye particles are substantially monodisperse.

19. The method of claim 14 wherein a radius of the colloidal dye particles have a radius in a range from about 10 nm to about 500 nm.

20. The method of claim 14 wherein the colloidal dye particles are substantially monodisperse.

21. The method of claim 14 wherein said solid phase is an interior surface of a liquid sample container, and wherein step c) includes drawing out liquid from said sample container.

22. The method of claim 14 wherein the solid-phase includes a plurality of magnetic particles, said magnetic particles being contained in a vessel, and wherein step c) includes using a magnetic field to enable separation of the coated colloidal dye particles not bound to said solid phase from the bound coated colloidal dye particle-target analyte complexes on the solid-phase.

23. The method of claim 22 including a step of controlling a surface area of the solid-phase by using a pre-selected amount of said magnetic particles.

24. The method of claim 22 including using a pre-selected amount of said magnetic particles for compensating for variations in properties of said dyes, including the affinity of bound receptors, and the smoothness, size and geometry of the dye particles.

25. The method of claim 14 including a step of extending a dynamic range of the assay by measuring an absorbance of said dye solution in addition to the fluorescence of said dye solution, and wherein said measured absorbance and fluorescence are related to a concentration of said target analyte in said known sample volume using pre-established standard curves.

26. A method for the detection of a target analyte, comprising the steps of:
a) contacting a solid-phase coated with receptors having a high affinity for the target analyte with a known volume of a liquid sample being tested for a presence or absence of the target analyte, the liquid sample containing a known amount of colloidal dye particles having the target analyte bound thereto, wherein in the absence of target analytes in the liquid sample target analytes bound to the colloidal dye particles bind to the receptors to form colloidal dye particle-target analyte-receptor complex, and in the presence of target analytes in the liquid sample the target analytes preferentially bind to the receptors to form target analyte-receptor complexes;
b) removing the solid phase from contact with said liquid sample and forming a dye solution by exposing the solid phase to a solubilizing solvent for solubilizing any dye particles of the colloidal dye particle-target analyte-receptor complexes into a solubilization buffer;
c) measuring fluorescence upon optically exciting said dye solution with excitation light at an appropriate wavelength; and
d) relating said measured fluorescence to a concentration of said target analyte in said known sample volume using a pre-established standard curve.

27. The method of claim 26 wherein said sample volume includes additional different types of target analytes, and wherein said solid-phase is coated with additional different types of receptors having a high affinity for the additional different target analytes so that said additional different types of target analytes bind with said additional different types of receptors so that said additional different types of target analytes are bound to said solid phase, and wherein said colloidal solution contains additional different types of colloidal dye particles coated with additional different target analytes so that said additional different types of target analytes compete with said additional different types of colloidal dye particles for binding sites of said additional types of receptors, and wherein forming a dye solution includes solubilizing the additional dye particles, and wherein measuring fluorescence includes exciting each additional dye in the dye solution with excitation light at an appropriate wavelength, and including relating said measured fluorescence to a concentration of each additional different type of target analyte in said known sample volume using pre-established standard curves.

28. The method of claim 26 wherein a radius of the colloidal dye particles have a radius in a pre-selected range.

29. The method of claim 27 wherein a radius of the additional colloidal dye particles have a radius in a pre-selected range.

30. The method of claim 27 wherein the additional types of colloidal dye particles are substantially monodisperse.

31. The method of claim 26 wherein a radius of the colloidal dye particles have a radius in a range from about 10 nm to about 500 nm.

32. The method of claim 26 wherein the colloidal dye particles are substantially monodisperse.

33. The method of claim 26 wherein said solid phase is an interior surface of a liquid sample container, and wherein step c) includes drawing out liquid from said sample container.

34. The method of claim 26 wherein the solid-phase includes a plurality of magnetic particles, said magnetic particles being contained in a vessel, and wherein step c) includes using a magnetic field to enable separation of the coated colloidal dye particles not bound to said solid phase from the bound coated colloidal dye particle-target analyte complexes on the solid-phase.

35. The method of claim 34 including a step of controlling a surface area of the solid-phase by using a pre-selected amount of said magnetic particles.

36. The method of claim 34 including using a pre-selected amount of said magnetic particles for compensating for variations in properties of said dyes, including the affinity of bound receptors, and the smoothness, size and geometry of the dye particles.

37. The method of claim 26 including a step of extending a dynamic range of the assay by measuring an absorbance of said dye solution in addition to the fluorescence of said dye solution, and wherein said measured absorbance and fluorescence are related to a concentration of said target analyte in said known sample volume using pre-established standard curves.

38. A method for the detection of a target analyte, comprising the steps of:
a) contacting a first solid-phase coated with receptors having a high affinity for the target analyte with a known volume of a liquid sample being tested for a presence or absence of the target analyte, the liquid sample containing a known amount of colloidal dye particles having the target analyte bound thereto, wherein in the absence of target analytes in the liquid sample target analytes bound to the colloidal dye particles bind to the receptors to form colloidal dye particle-target analyte-receptor complex, and in the presence of target analytes in the liquid sample the target analytes preferentially bind to the receptors to form target analyte-receptor complexes;
b) separating said coated colloidal dye particles not bound to said solid phase from the bound coated colloidal dye particle-target analyte complexes on the solid-phase;
c) forming a dye solution by solubilizing the dye particles not bound to said solid phase into a solubilization buffer which is maintained in a pre-selected pH range;
d) measuring fluorescence upon optically exciting said dye solution with excitation light at an appropriate wavelength; and
e) relating said measured fluorescence to a concentration of said target analyte in said known sample volume using a pre-established standard curve.

39. The method of claim 38 wherein said sample volume includes additional different types of target analytes, and wherein said solid-phase is coated with additional different types of receptors having a high affinity for the additional different target analytes so that said additional different types of target analytes bind with said additional different types of receptors so that said additional different types of target analytes are bound to said solid phase, and wherein said colloidal solution contains additional different types of colloidal dye particles coated with additional different target analytes so that said additional different types of target analytes compete with said additional different types of colloidal dye particles for binding sites of said additional types of receptors, and wherein forming a dye solution includes solubilizing the additional dye particles, and wherein measuring fluorescence includes exciting each additional dye in the dye solution with excitation light at an appropriate wavelength, and including relating said measured fluorescence to a concentration of each additional different type of target analyte in said known sample volume using pre-established standard curves.

40. The method of claim 38 wherein a radius of the colloidal dye particles have a radius in a pre-selected range.

41. The method of claim 38 wherein a radius of the colloidal dye particles have a radius in a range from about 10 nm to about 500 nm.

42. The method of claim 38 wherein the colloidal dye particles are substantially monodisperse.

43. The method of claim 38 wherein said solid phase is an interior surface of a liquid sample container, and wherein step c) includes drawing out liquid from said sample container.

44. The method of claim 38 wherein the solid-phase includes a plurality of magnetic particles, said magnetic particles being contained in a vessel, and wherein step c) includes using a magnetic field to enable separation of the coated colloidal dye particles not bound to said solid phase from the bound coated colloidal dye particle-target analyte complexes on the solid-phase.

45. The method of claim 44 including a step of controlling a surface area of the solid-phase by using a pre-selected amount of said magnetic particles.

46. The method of claim 44 including using a pre-selected amount of said magnetic particles for compensating for variations in properties of said dyes, including the affinity of bound receptors, and the smoothness, size and geometry of the dye particles.

47. The method of claim 38 including a step of extending a dynamic range of the assay by measuring an absorbance of said dye solution in addition to the fluorescence of said dye solution, and wherein said measured absorbance and fluorescence are related to a concentration of said target analyte in said known sample volume using pre-established standard curves.

48. The method of claim 39 wherein a radius of the additional colloidal dye particles have a radius in a pre-selected range.

49. The method of claim 39 wherein the additional types of colloidal dye particles are substantially monodisperse.
